(19) United States
(12) Patent Application Publication
(10) Pub. No.: US 2015/0112108 A1
(43) Pub. Date: Apr. 23, 2015

(54) METHOD FOR PREPARING HIGH-YIELD ADAMANTANE

(71) Applicant: NATIONAL CHUNG SHAN INSTITUTE OF SCIENCE AND TECHNOLOGY, LONGTAN TOWNSHIP (TW)

(72) Inventors: CHI-FA HSIEH, DAXI TOWNSHIP (TW); SHU-CHEN CHIANG, TOUFEN TOWNSHIP (TW); WEN-CHIUNG SU, TAIPEI CITY (TW); FU-YENG LIN, LONGTAN TOWNSHIP (TW)

(73) Assignee: NATIONAL CHUNG SHAN INSTITUTE OF SCIENCE AND TECHNOLOGY, LONGTAN TOWNSHIP (TW)

(21) Appl. No.: 14/279,379

(22) Filed: May 16, 2014

(30) Foreign Application Priority Data
Oct. 18, 2013 (TW) 102137595

(51) Int. Cl. C07C 5/27

(52) U.S. Cl. CPC C07C 5/2727; C07C 2103/74; C07C 2531/26
USPC 585/352

(57) ABSTRACT

A method prepares high-yield adamantane in the presence of an ionic liquid catalyst formed by mixing an aluminum bromide-containing or aluminum iodide-containing catalyst and an ionic liquid. Given the method, isomerization of tetrahydrodicyclopentadiene (THDCPD) takes place in the presence of the ionic liquid catalyst to produce adamantane of high reactant conversion, high selectivity, and high yield and cut costs.

### CROSS-REFERENCE TO RELATED APPLICATION

This non-provisional application claims priority under 35 U.S.C. §119(a) on Patent Application No(s). 102137595 filed in Taiwan, R.O.C. on Oct. 18, 2013, the entire contents of which are hereby incorporated by reference.

### FIELD OF TECHNOLOGY

The present invention relates to methods for preparing high-yield adamantane, and more particularly, to a method for preparing high-yield adamantane, characterized in that isomerization is performed in the presence of an aluminum bromide-containing or aluminum iodide-containing ionic liquid catalyst to produce adamantane of high reactant conversion, high selectivity, and high yield.

### BACKGROUND

According to the prior art, various chemicals, which are raw materials for use in the production of gasoline, diesel, fuel oil, and petrochemicals, are produced in different stages of the naphtha pyrolysis and oil refining processes carried out at the onset of the processing of crude oil. However, among conventional products of oil refining, five-carbon chemicals are not put to good use due to limit of yield, as the five-carbon chemicals are mostly re-processed to produce gasoline and diesel for sales at low prices.

Among the aforesaid five-carbon chemicals, dicyclopentadiene (DCPD) has a double bond and thus can undergo chemical reactions to produce plenty of products. One of the products serves as a high-performance epoxy resin. Another product undergoes polymerization ethylene and propylene to produce polymeric rubbers which in turn function as the raw materials for use in the production of plenty of rubber-made parts and components. Yet another product is produced from polymerization of DCPD to serve as industrial plastic raw materials for use in the manufacturing of the parts and components of vehicles, transportation-related electrical apparatuses, communication-related apparatuses, and storage tanks.

DCPD undergoes hydrogenation and isomerization in the presence of a catalyst to produce endo-tetrahydrodicyclopentadiene (endo-THDCPD) and exo-tetrahydrodicyclopentadiene (exo-THDCPD). As exo-THDCPD has excellent heat of combustion and low-temperature properties (flash point, melting point, and viscosity), it is currently a required high-energy fuel for use in aerospace engineering. Hydrogenated DCPD also serves as an intermediate product which undergoes a catalytic reaction to produce adamantane (ADM)—a symmetric, stable, and cage hydrocarbon dubbed as “new-generation special chemical engineering material”, and its group derivatives are applied widely to special functional material (resin with high index of refraction and low dielectric constant and for use in packaging, and resin with low dielectric constant and low thermal conductivity and for use in flame retardation), special photovoltaic materials (photoresist, and high-level plastic optical fiber), and drugs (for treating viral infection, cancer, Parkinson's disease, and hypertension).

Conventional methods for preparing ADM fall within the following three categories: aluminum chloride (AlCl₃)-catalyzed isomerization; using an ultra-strong acid, such as HF catalyst and BF3 catalyst; and zeolite molecular sieve isomerization. In this regard, zeolite molecular sieve isomerization requires rare-earth metals and noble metals, has a low ADM yield, and is disadvantaged by an intricate zeolite preparation process. When used as a catalyst, aluminum chloride, HF, and BF3 render the preparation process simple but produce an overly large number of products which are difficult to separate, not to mention that aluminum chloride, HF, and BF3 are disadvantaged by a high dosage requirement, a short service life, a lack of readiness for recycling and reuse, the need to undergo special treatment before being discharged to the environment, their being highly corrosive to apparatuses, and their being environment unfriendly.

Recent decades see the advent of a more environment-unfriendly ADM preparation method which entails providing an ionic liquid for use as a carrier and introducing aluminum chloride for use as an ionic liquid catalyst. Conventional ionic liquids, which are ionic compounds in the form of liquids below 100° C. and still manifest ionic properties, function as a solvent for use in catalytic reactions over the past decades, wherein aluminum chloride is introduced to function as an ionic liquid catalyst for use in isomerization to prepare ADM, as disclosed in U.S. Pat. No. 7,488,859 and Taiwan Patent 1321128, wherein aluminum chloride is introduced to function as the ionic liquid catalyst for use in preparing ADM with a high yield of 21.9%. Conventional ADM preparation methods for use in mass production employ aluminum chloride which functions as a catalyst, in the presence of the catalyst exo-tetrahydrodicyclopentadiene (exo-THDCPD) undergoes isomerization to produce ADM. Products of the isomerization have the same molecular formula but differ in the arrangement of atoms. Nonetheless, it is impossible for the combination of the prior art pertaining to the aforesaid conventional preparation methods and an ionic liquid catalyst which contains an aluminum chloride-containing catalyst and operates with an augmented concentration of the aluminum chloride to achieve high reactant conversion and high selectivity and eventually high ADM yield.

### SUMMARY

In view of the aforesaid drawbacks of the prior art, it is an objective of the present invention to provide a method for preparing high-yield adamantane (ADM). The method is characterized in that: a catalyst, which contains aluminum bromide or aluminum iodide, mixes with an ionic liquid to form an ionic liquid catalyst, and isomerization of tetrahydrodicyclopentadiene (THDCPD) takes place in the presence of the ionic liquid catalyst to produce ADM of high reactant conversion, high selectivity, and high yield.

Conventional aluminum chloride cannot produce ADM with a high yield. An ionic liquid catalyst is rarely homogenously liquid below 100° C. according to the prior art, because of limit of related chemical knowledge and expertise, limit of its physical and chemical properties, strict reactivity requirements, and inadequate understanding of reactivity mechanism. Hence, the method of the present invention entails providing aluminum bromide (AlBr₃) or aluminum iodide of a specific melting point as a catalyst. Take aluminum bromide as an example, it not only destroys the crystalline stacking of the ionic liquid but also forms an ionic liquid catalyst with high aluminum bromide content by reacting with an ionic liquid in the absence of any carrier or any other auxiliary solvent. The ionic liquid catalyst with high aluminum bromide content is homogenously liquid and thus effectively meets the need of easy separation of the ionic liquid catalyst and products as well as the recycling and reuse of the ionic liquid catalyst. The ionic liquid catalyst, which forms as a result of the reaction of the ionic liquid with aluminum chloride whose content equals the aluminum bromide content, still comes in the form of aluminum chloride powder. The ionic liquid catalyst is in solid-liquid dual phases and forms the ionic liquid catalyst identical to one formed solely with aluminum chloride powder; hence, the ionic liquid catalyst has the following disadvantages: it is difficult to separate from the other products, it cannot be recycled and reused and thus is unfriendly to the environment, and it incurs high costs. In view of this, to obtain high-yield ADM, it is necessary to switch to the other catalysts rather than increase the concentration of aluminum chloride.

The ionic liquid provided by the present invention is a quaternary phosphonium halide, a quaternary ammonium halide, or a combination thereof, wherein the quaternary phosphonium halide is exemplified by tetraalkylphosphonium halide. The quaternary ammonium halide is exemplified by trialkylpyrrolidinium halide, trialkylpyrazinium halide, trialkylpyrimidinium halide, tetraalkylammonium halide, trialkylimidazolium halide, trialkylpyridazinium halide, dialkylpyridinium halide, or trialkylpiperidinium halide. The molar ratio of catalyst aluminum bromide (AlBr₃) or aluminum iodide (AlI₃) to the ionic liquid is 0.1˜8.0.

THDCPD, a reactant of the present invention, is either endo-tetrahydrodicyclopentadiene (endo-THDCPD) or exo-tetrahydrodicyclopentadiene (exo-THDCPD). The molar ratio of aluminum bromide (AlBr₃) or aluminum iodide (AlI₃) of the ionic liquid catalyst to the THDCPD is 0.1˜8.0. The reaction duration of the isomerization process ranges from 0.5 hour to 24 hours. The isomerization process takes place at 50˜100° C.

The molecular configuration of the endo-THDCPD, exo-THDCPD, and ADM are depicted below. THDCPD undergoes the isomerization process to produce ADM.

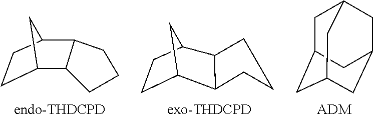

The process of preparing the aluminum bromide-containing or aluminum iodide-containing ionic liquid catalyst according to the present invention is simple and convenient, as it merely requires mixing the ionic liquid and a catalyst in a moisture-free environment at room temperature to produce an ionic liquid catalyst which comes in the form of a homogenous liquid, thereby dispensing with any solvent or carrier and thus subsequent treatment, causing no environmental pollution, enabling recycling, and cutting production costs. Last but not least, the method of the present invention is further advantageously characterized in that, despite an increase in the concentration of the catalyst, the ionic liquid catalyst remains a homogenous liquid conducive to multiple instances of recycling, thereby cutting production costs.

### DETAILED DESCRIPTION OF THE PREFERRED EMBODIMENTS

Objectives, features, and advantages of the present invention are hereunder illustrated with specific embodiments.

The present invention provides a method for preparing high-yield adamantane (ADM). The method of the present invention involves using tetrahydrodicyclopentadiene (THDCPD) as a reactant and performing isomerization on THDCPD in the presence of an appropriate ionic liquid catalyst to produce ADM with a high yield. The raw material for use in the isomerization is endo-tetrahydrodicyclopentadiene (endo-THDCPD) which, in the initial stage of the isomerization, is mostly isomerized to become exo-tetrahydrodicyclopentadiene (exo-THDCPD) which continues with the isomerization in the presence of the same ionic liquid catalyst to produce the end product, that is, ADM. In general, endo-THDCPD of low purity comes in the form of a liquid and thus dispenses with any solvent. Alternatively, as endo-THDCPD has a low melting point, it mixes well with the ionic liquid catalyst during the isomerization. In case the endo-THDCPD in use is of high purity, it is in a solid state at room temperature; to increase the chance that the solid-state endo-THDCPD comes into contact with the ionic liquid catalyst, it is necessary to dissolve the solid-state endo-THDCPD in an appropriate solvent, such as cyclohexane or exo-THDCPD (in addition to its role as an intermediate product as mentioned earlier, exo-THDCPD serves as a solvent for the solid-state endo-THDCPD). Nonetheless, an appropriate ionic liquid catalyst is the key to enabling the isomerization to demonstrate high reactant conversion and high selectivity. The reactant conversion and selectivity of the isomerization disclosed in the present invention are determined by performing data analysis by GC-MASS. The reactant conversion of the endo-THDCPD is the rate at which endo-THDCPD vanishes. ADM selectivity refers to the proportion of ADM out of the products. ADM yield is calculated by multiplying endo-THDCPD reactant conversion by ADM selectivity.

A wide variety of ionic liquids are suitable for use in preparing the ionic liquid catalyst, including ammonium, guanidinium, imidazole, trisubstituted imidazole, isouroium/thiouronium, phosphonium, and pyridine. Depending on the environment of reaction, the ionic liquid is chosen according to such physical properties as its melting point or whether it is hydrophilic or lipophilic. The method of the present invention requires a non-conventional catalyst, such as aluminum bromide or aluminum iodide, which reacts with the ionic liquid at an appropriate proportion to prepare an ionic liquid catalyst. At the beginning of the process of preparing the ionic liquid catalyst, the raw materials of the ionic liquid catalyst are weighed in a dry environment (for example, a closed space with a desiccant therein), otherwise the ensuing isomerization will be compromised by moisture. The process of mixing the ionic liquid and the catalyst is exothermic; hence, to be safe, it is necessary to add the catalyst slowly and piecemeal. The process of mixing the ionic liquid and the catalyst takes place at 25˜60° C., that is, at room temperature or in a slightly heated up environment. Mixing the ionic liquid and the catalyst causes them to melt and end up in liquid state. An ionic liquid catalyst with high solubility is a transparent liquid at room temperature.

To prepare ADM, it is necessary for the ionic liquid catalyst to react with an appropriate amount of a reactant, such as endo-THDCPD. If necessary, a solvent, such as cyclohexane or exo-THDCPD, is included. Since one of the reaction requirements of isomerization is being moisture-free, the chemicals weighing process and feeding process must be carried out in a dry environment or in stages by means of a pump for feeding the raw materials to a reactor. The process of isomerization is placed under the control of a temperature controlling circuit, wherein a temperature sensor is mounted inside a reactor. Depending on the size of the reactor, the process of isomerization necessitates mechanical or magnet-based blending and takes place at 60˜80° C. and preferably 70° C. for 0.5˜4 hours and preferably 2.5 hours, such that ADM is prepared in the presence of the aluminum bromide-containing ionic liquid catalyst

### Embodiment 1

Weigh, in a moisture-free closed box, 5.2 grams (0.0325 mole) of PHB (Pyridine hydrobromide) and put the PHB in a single-mouth round-base flask. Add 8,67 grams (0.0650 mole) of aluminum chloride into the PHB in the flask, and stir the mixture at room temperature such that the two solids melt down to miscible liquids while heat is being released, thereby resulting in the ionic liquid catalyst. Then, add 5.24 grams (0.0385 mole) of endo-THDCPD and 5.24 grams of n-hexane (i.e., the solvent) into the liquids, mount the temperature sensor in place, and remove the flask from the closed box, before putting the closed box in an oil bath at 70° C. Then, allow the aforesaid reactants to undergo isomerization at the constant temperature for 2.5 hours. Regarding the aforesaid reactants, the molar ratio of endo-THDCPD to PHB is 1.185, the molar ratio of endo-THDCPD to aluminum chloride is 0.59, the molar ratio of aluminum chloride to PHB is 2.0, and thus the mole fraction of aluminum chloride to the PHB in the ionic liquid catalyst is 0.67. At the end of the isomerization process, its products are analyzed by GC-MASS to thereby calculate and determine that the reactant conversion is equal to 26.12%, the selectivity of ADM equal to 72.17%, and the yield of ADM equal to 18.85%.

### Embodiment 2

In embodiment 2, the molar ratio of aluminum chloride to PHB is 3.0, the mole fraction of aluminum chloride to the PHB in the ionic liquid catalyst is 0.75, and the molar ratio of endo-THDCPD to aluminum chloride is 0.39. At the end of the isomerization process, its products are analyzed by GC-MASS to calculate and determine that the reactant conversion is equal to 65.19%, the selectivity of ADM equal to 42.77%, and the yield of ADM equal to 27.88%. During the reaction process, aluminum chloride solid powder always exists because of a surplus of aluminum chloride; as a result, it is difficult to separate the ionic liquid catalyst and the products and impossible to recycle and reuse the ionic liquid catalyst, thereby adding to the production cost.

### Embodiment 3

In embodiment 3, aluminum bromide is in use, the molar ratio of endo-THDCPD to PHB is 1.185, the molar ratio of endo-THDCPD to aluminum bromide is 0.59, the molar ratio of aluminum bromide to PHB is molar ratio 2.0, and thus the mole fraction of aluminum bromide to the PHB in the ionic liquid catalyst is 0.67. The resultant ionic liquid catalyst comes in the form of a transparent liquid, and the isomerization process takes place at a constant temperature of 70° C.° for 2.5 hours. At the end of the isomerization process, its products are analyzed by GC-MASS to calculate and determine that the reactant conversion is equal to 37.30%, the selectivity of ADM equal to 75.45%, and the yield of ADM equal to 28.14%.

### Embodiment 4

In embodiment 4, the molar ratio of aluminum bromide to PHB is 3.0, and thus the mole fraction of aluminum bromide to the PHB in the ionic liquid catalyst s 0.75, wherein the weight ratio of endo-THDCPD to cyclohexane is 0.4. The isomerization process takes place at a constant temperature of 70° C. The resultant ionic liquid catalyst comes in the form of a transparent liquid. At the end of the isomerization process, its products are analyzed by GC-MASS to calculate and determine that the reactant conversion is equal to 92.12%, the selectivity of ADM equal to 60.48%, and the yield of ADM equal to 55.72%.

### Embodiment 5

Embodiment 5 is different from embodiment 4 in that, in embodiment 5, the ionic liquid is fed by dropping. At he end of the isomerization process, its products are analyzed by GC-MASS to calculate and determine that the reactant conversion is equal to 46.96%, the selectivity of ADM equal to 58.57%, and the yield of ADM equal to 27.51%.

### Embodiment 6

Embodiment 6 is different from embodiment 4 in that, in embodiment 6, the isomerization process takes place at 80° C. At the end of the isomerization process, its products are analyzed by GC-MASS to calculate and determine that the reactant conversion is equal to 65.38%, the selectivity of ADM equal to 46.09%, and the yield of ADM equal to 30.14%.

### Embodiment 7

Embodiment 7 is different from embodiment 2 in that: in addition to the raw materials used in embodiment 2, embodiment 7 requires aluminum bromide, and embodiment 7 entails adding 5.2 grams (0.0325 mole) of PHB to 4.34 grams (0.0163 mole) of aluminum bromide, and adding 13 grams (0.0975 mole) of aluminum chloride, so as to form the ionic liquid catalyst, and at this point in time, the ionic liquid catalyst comes in he from of solid-liquid dual phases, afterward, 5.24 grams (0.0385 mole) of endo-THDCPD and 13.1 grams of cyclohexane (i.e. the solvent) are added, such that the weight ratio of endo-THDCPD to cyclohexane is 0.4, the molar ratio of endo-THDCPD to PHB is 1.185, the molar ratio of endo-THDCPD to aluminum chloride is 0.39, the molar ratio of aluminum chloride to PHB is 3.0, and thus the mole fraction of aluminum chloride to the ionic liquid catalyst is 0.75, and the molar ratio of aluminum bromide to PHB is 0.5, and thus the mole fraction of aluminum bromide to the PHB in the ionic liquid catalyst is 0.33. The isomerization process takes place at a constant temperature of 70° C. for 2.5 hours. At the end of the isomerization process, its products are analyzed by GC-MASS to calculate and determine that he reactant conversion is equal to 79.04%, the selectivity of ADM equal to 41.91%, and the yield of ADM equal to 33.12%.

### Embodiment 8

Unlike embodiment 4 which employs cyclohexane, embodiment 8 employs exo-THDCPD and yields the ionic liquid catalyst in the form of a homogeneous liquid. At the end of the isomerization process, its products are analyzed by GC-MASS to calculate and determine that the reactant conversion is equal to 40.25%, the selectivity of ADM equal to 96.64%, and the yield of ADM equal to 38.89%. Afterward, the ionic liquid catalyst is recycled to perform the first experiment which entails taking upper products, adding aforesaid raw materials thereto, sampling the mixture to create the basis of calculation of products of the experiment, and placing the mixture in an oil bath. At the end of the isomerization process, its products are analyzed by GC-MASS to calculate and determine that the reactant conversion is equal to 27.49%, the selectivity of ADM equal to 94.30%, and the yield of ADM equal to 25.99%. Like the first experiment performed with the ionic liquid catalyst recycled, the second experiment and the third experiment are performed. In Table 1, the first instance of recycling and reusing the ionic liquid catalyst is denoted with the words “reuse 1”, the second instance of recycling and reusing the ionic liquid catalyst is denoted with the words “reuse 2”, and the third instance of recycling and reusing the ionic liquid catalyst is denoted with the words “reuse 3”. In the second instance of recycling and reusing the ionic liquid catalyst, the reactant conversion is equal to 18.84%, the selectivity of ADM equal to 91.44%, and the yield of ADM equal to 17.23%. In the third instance of recycling and reusing the ionic liquid catalyst, the reactant conversion is equal to 14.30%, the selectivity of ADM equal to 88.06%, and the yield of ADM equal to 12.59%.

Data required for and generated from the aforesaid embodiments are shown in Table 1.

TABLE 1
data required for and generated from embodiments 1~8

| | | | | Component in ionic | | | | |
|---|---|---|---|---|---|---|---|---|
| | | | | liquid of PHB | | | | |
| | | | | Molar | Molar | | | |
| | | | Temp. | fraction | fraction | Conversion | Selectivity | Productivity |
| Exp. | Reactant | Solvent | (C. °) | of AlCl₃ | of AlBr₃ | of reactant | of ADM | of ADM |
| 1 | endo- | Cyclo | 70 | 0.67 | — | 26.12 | 72.17 | 18.85 |
| | THDCPD | hexane | | | | | | |
| 2 | endo- | Cyclo | 70 | 0.75 | — | 65.19 | 42.77 | 27.88 |
| | THDCPD | hexane | | | | | | |
| 3 | endo- | Cyclo | 70 | — | 0.67 | 37.30 | 75.45 | 28.14 |
| | THDCPD | hexane | | | | | | |
| 4 | endo- | Cyclo | 70 | — | 0.75 | 92.12 | 60.48 | 55.72 |
| | THDCPD | hexane | | | | | | |
| 5 | endo- | Cyclo | 70 | — | 0.75 | 46.96 | 58.57 | 27.51 |
| | THDCPD | hexane | | | (dropping) | | | |
| 6 | endo- | Cyclo | 80 | — | 0.75 | 65.38 | 46.09 | 30.14 |
| | THDCPD | hexane | | | | | | |
| 7 | endo- | Cyclo | 70 | 0.75 | 0.333 | 79.04 | 41.91 | 33.12 |
| | THDCPD | hexane | | | | | | |
| 8 | endo- | exo- | 70 | — | 0.75 | 40.25 | 96.64 | 38.89 |
| | THDCPD | THDCPD | | | | | | |
| | endo- | exo- | 70 | — | reuse 1 | 27.49 | 94.30 | 25.92 |
| | THDCPD | THDCPD | | | | | | |
| | endo- | exo- | 70 | — | reuse 2 | 18.84 | 91.44 | 17.23 |
| | THDCPD | THDCPD | | | | | | |
| | endo- | exo- | 70 | — | reuse 3 | 14.30 | 88.06 | 12.59 |
| | THDCPD | THDCPD | | | | | | |

As revealed by Table 1, under the same condition, the isomerization of endo-THDCPD is advantageously characterized in that the aluminum bromide-containing ionic liquid catalyst surpasses the conventional aluminum chloride-containing ionic liquid catalyst in producing ADM with a high yield. Furthermore, given the mole fraction of 0.67 of aluminum chloride to the PHB in the ionic liquid catalyst (in embodiment 1 and embodiment 3), the yield of ADM produced in the presence of the aluminum bromide-containing ionic liquid catalyst is 28.14%, but the yield of ADM produced in the presence of the conventional aluminum chloride-containing ionic liquid catalyst is 18.85%. Moreover, given the mole fraction of 0.75 of aluminum chloride to the PHB in he ionic liquid catalyst (in embodiment 2 and embodiment 4), the yield of ADM produced in the presence of the aluminum bromide-containing ionic liquid catalyst is 55.72%, but the yield of ADM produced in the presence of the conventional aluminum chloride-containing ionic liquid catalyst is 27.88%, wherein aluminum chloride solid powder is still found in the conventional aluminum chloride-containing ionic liquid catalyst, hereby rendering it difficult to separate the ionic liquid catalyst from the products. Hence, at different catalyst molar ratios, the method for preparing high-yield ADM according to the present invention produces ADM with a higher yield than the conventional ADM preparation methods using aluminum chloride-containing ionic liquid catalysts, and thus the method of present invention involves an inventive step.

The present invention is disclosed above by preferred embodiments. However, persons skilled in the art should understand that the preferred embodiments are illustrative of the present invention only, but should not be interpreted as restrictive of the scope of the present invention. Hence, all equivalent modifications and replacements made to the aforesaid embodiments should fall within the scope of the present invention. Accordingly, the legal protection for he present invention should be defined by the appended claims.

What is claimed is:

1. A method for preparing high-yield adamantane (ADM), the method comprising the steps of:
(A) providing aluminum bromide (AlBr₃) or aluminum iodide (AlI₃) as a catalyst for mixing with an ionic liquid to form the ionic liquid catalyst;
(B) mixing the ionic liquid catalyst and tetrahydro-dicyclopentadiene (THDCPD) to form a reacting solution; and
(C) performing isomerization on the reacting solution to prepare ADM.

2. The method of claim 1, wherein the ionic liquid in the step (A) is one selected from the group consisting of a quaternary phosphonium halide, a quaternary ammonium halide, and a combination thereof.

3. The method of claim 2, wherein the quaternary phosphonium halide is exemplified by tetraalkylphosphonium halide.

4. The method of claim 2, wherein the quaternary ammonium halide is one selected from the group consisting of trialkylpyrrolidinium halide, trialkylpyrazinium halide, trialkylpyrimidinium halide, tetraalkylammonium halide, trialkylimidazolium halide, trialkylpyridazinium halide, dialkylpyridinium halide, trialkylpiperidinium halide, and a combination thereof.

5. The method of claim 2, wherein the quaternary ammonium halide is one selected from the group consisting of Pyridine hydrobromide (PHB), pyridine hydrochloride (PHC), tetraethylammonium chloride (TEAL), 1-butyl-3-methylimidazolium chloride (BMIC), and a combination thereof.

6. The method of claim 1, wherein, in the step (A), molar ratio of aluminum bromide (AlBr₃) or aluminum iodide (AlI₃) to the ionic liquid is 0.1˜8.0.

7. The method of claim 1, wherein the mixing in the step (A) takes place in a moisture-free environment.

8. The method of claim 1, wherein the THDCPD in the step (B) is exemplified by one of endo-THDCPD and exo-THDCPD.

9. The method of claim 1, wherein, in the step (B), molar ratio of aluminum bromide (AlBr₃) or aluminum iodide (AlI₃) of the ionic liquid catalyst to the THDCPD is 0.1˜8.0.

10. The method of claim 1, wherein, in the step (B), the reacting solution further comprises a solvent.

11. The method of claim 1, wherein the isomerization of the step (C) takes place at 50˜100° C.

12. The method of claim 1, wherein the isomerization of the step (C) takes 0.5˜24 hours.

13. The method of claim 1, wherein the isomerization of the step (C) is followed by the step of recycling the ionic liquid catalyst from the reacting solution and performing steps (A)18 (C) with the recycled ionic liquid catalyst to prepare ADM.
